SEQUENCE LISTING (4 sequences)

SEQ ID NO: 1 - DNA, 19 bp, Artificial Sequence
  A synthetic PCR primer 1492R
ggttaccttgttacgactt

SEQ ID NO: 2 - DNA, 20 bp, Artificial Sequence
  A synthetic PCR primer F27
agagtttgatcctggctcag

SEQ ID NO: 3 - DNA, 1413 bp, Enterobacter FDS8
  misc_feature: 16S rDNA
agtggtaagcgccctcccgaaggttaagctacctacttcttttgcaacccactcccatggtgtgacgggcggtgtgtacaaggcccgggaacgtattcaccgtggcattctgatccacgattactagcgattccgacttcatggagtcgagttgcagactccaatccggactacgacatactttatgaggtccgcttgctctcgcgaggtcgcttctctttgtatatgccattgtagcacgtgtgtagccctggtcgtaagggccatgatgacttgacgtcatccccaccttcctccagtttatcactggcagtctcctttgagttcccggccggaccgctggcaacaaaggataagggttgcgctcgttgcgggacttaacccaacatttcacaacacgagctgacgacagccatgcagcacctgtctcacggttcccgaaggcactaaggcatctctgccaaattccgtggatgtcaagaccaggtaaggttcttcgcgttgcatcgaattaaaccacatgctccaccgcttgtgcgggcccccgtcaattcatttgagttttaaccttgcggccgtactccccaggcggtcgacttaacgcgttagctccggaagccacgcctcaagggcacaacctccaagtcgacatcgtttacggcgtggactaccagggtatctaatcctgtttgctccccacgctttcgcacctgagcgtcagtctttgtccagggggccgccttcgccaccggtattcctccagatctctacgcatttcaccgctacacctggaattctacccccctctacaagactcaagcctgccagtttcgaatgcagttcccaggttgagcccggggatttcacatccgacttgacagaccgcctgcgtgcgctttacgcccagtaattccgattaacgcttgcaccctccgtattaccgcggctgctggcacggagttagccggtgcttcttctgcgggtaacgtcaatcaacacggttattaaccgtattgccttcctccccgctgaaagtgctttacaacccgaaggccttcttcacacacgcggcatggctgcatcaggcttgcgcccattgtgcaatattccccactgctgcctcccgtaggagtctggaccgtgtctcagttccagtgtggctggtcatcctctcagaccagctagggatcgtcgccttggtgagccattacctcaccaactagctaatcccatctgggcacatccgatggcaagaggcccgaaggtccccctctttggtcttgcgacattatgcggtattagctaccgtttccagtagttatccccctccatcgggcagtttcccagacattactcacccgtccgccactcgtcacccgagagcaagctctctgtgctaccgttcgacttgca

SEQ ID NO: 4 - DNA, 1433 bp, Bacillus ADS3
  misc_feature: 16S rDNA
ccttcggcggctggctccaaaaggttacctcaccgacttcgggtgttacaaactctcgtggtgtgacgggcggtgtgtacaaggcccgggaacgtattcaccgcggcatgctgatccgcgattactagcgattccggcttcatgtaggcgagttgcagcctacaatccgaactgagaacgactttatcggattagctccctctcgcgagttggcaaccgtttgtatcgtccattgtagcacgtgtgtagcccaggtcataaggggcatgatgatttgacgtcatccccaccttcctccggtttgtcaccggcagtcaccttagagtgcccaactaaatgatggcaactaagatcaagggttgcgctcgttgcgggacttaacccaacatctcacgacacgagctgacgacaaccatgcaccacctgtcaccgttgtccccgaagggaaaaccatatctctacagtggtcaacgggatgtcaagacctggtaaggttcttcgcgttgcttcgaattaaaccacatgctccaccgcttgtgcgggcccccgtcaattcctttgagtttcagtcttgcgaccgtactccccaggcggagtgcttaatgcgttagctgcagcactaaggggcggaaaccccctaacacttagcactcatcgtttacggcgtggactaccagggtatctaatcctgtttgctccccacgctttcgcgcctcagtgtcagttacagaccagatagtcgccttcgccactggtgttcctccaaatctctacgcatttcaccgctacacttggaattccactatcctcttctgcactcaagtctcccagtttccaatgaccctccacggttgagccgtgggctttcacatcagacttaagaaaccacctgcgcgcgctttacgcccaataattccggacaacgcttgccacctacgtattaccgcggctgctggcacgtagttagccgtggctttctaataaggtaccgtcaaggtacagccagttactactgtacttgttcttcccttacaacagagttttacgaaccgaaatccttcttcactcacgcggcgttgctccatcaggctttcgcccattgtggaagattccctactgctgcctcccgtaggagtctgggccgtgtctcagtcccagtgtggccgatcaccctctcaggtcggctacgcatcgtcgccttggtgagccgttacctcaccaactagctaatgcgccgcgggcccatcctatagcgacagccgaaaccgtctttcagtattgtcccatgagggacaatagattattcggtattagccccggtttcccggagttatcccaaactataaggtaggttgcccacgtgttactcacccgtccgccgctaacgtcaaaggagcaagctccttctctgttcgctcgacttgcatgtatag

(19) United States
(12) Patent Application Publication
(10) Pub. No.: US 2015/0118736 A1
(43) Pub. Date: Apr. 30, 2015

(54) METHOD OF DEGRADING ORGANIC COMPOUNDS

(71) Applicant: Agency for Science, Technology and Research, Singapore (SG)

(72) Inventors: Jin Chuan Wu, Jurong Island (SG); Dongxu Zhang, Jurong Island (SG); Yee Ong, Jurong Island (SG)

(21) Appl. No.: 14/397,365

(22) PCT Filed: Apr. 29, 2013
(86) PCT No.: PCT/SG2013/000170

§ 371 Date: Oct. 27, 2014

(30) Foreign Application Priority Data
Apr. 27, 2012 (SG) 201203160-5

(51) Int. Cl. C12P 17/04

(52) U.S. Cl. CPC C12P 17/04
USPC 435/276

(57) ABSTRACT

There is provided a method of degrading a heterocyclic aldehyde compound comprising the step of treating the heterocyclic aldehyde compound with Enterobacter sp. microorganisms. There is also provided a method of degrading a carboxylic acid compound comprising the step of treating the carboxylic acid compound with Bacillus sp. microorganisms.

### TECHNICAL FIELD

The present invention generally relates to a method of degrading a heterocyclic aldehyde compound. The present invention also relates to a method of degrading a carboxylic acid compound.

### BACKGROUND

Lignocellulosic materials such as wood are a natural and abundant source of renewable energy. These materials contain polymerized sugars in the form of cellulose and hemicellulose. These polymerized sugars are fermentable, and may be liberated by subjecting the lignocellulosic materials to hydrolytic processes.

Subsequently the sugars can be fermented to an alcohol by microorganisms such as Saccharomyces cerevisiae, Thermoanaerobacterium strain AK17, Caldicellulosiruptor saccharolyticus and Thermotoga neapolitana and Pseudomaonas sppm.

Many degradation products such as hydroxymethyl furfural (HMF), furfural, acetic acid, formic acid, ferulic acid, vanillin, 4-hydroxybenzaldehyde, guaiacol and phenol are known to inhibit biodegradation reactions, damage the cell membranes of the microorganisms, or reduce cell viability through the interference with essential physiological processes of the microorganisms.

Microorganisms that participate in the degradation of lignocellulosic materials therefore face the challenges of having to provide a desirable yield of biofuels, and basic survival in an environment containing toxic substances.

Among the degradation products, furfural, HMF and acetic acid are generally considered the most harmful to microbial strains introduced, in view of their pronounced presence in the hydrolysate products of lignocellulose.

While various methods such as over-liming, adsorption with active charcoal, ion exchange and enzymatic treatments have been proposed for the removal or degradation of the inhibitory compounds from lignocellulose hydrolysate to favor microbial fermentation, these methods are usually commercially unattractive due to the relatively high process costs stemming from complicated operations or the generation of large amount of wastes.

An alternative method for removing the inhibitory compounds described earlier comes in the form of biological detoxification. Biological detoxification presents the advantages of requiring only straightforward processes, and generally generates less waste material to be disposed of.

However, the efficiency of biological detoxification is usually low. For examples, the highest rates of degrading furfural, HMF and acetic acid by biological detoxification were respectively reported to be 0.1, 0.02 and 0.27 g/L/h. In addition, the detoxification process significantly requires long periods of up to 4 days to be completed. The generally low efficiencies of biological degradation severely limit its applications in industry.

Accordingly, there is a need to provide suitable microbiological strains to provide the desired efficiency of biological detoxification while being able to resist inhibitory stresses.

There is a need to provide a method for degrading an organic compound that overcomes, or at least ameliorates, one or more of the disadvantages described above.

### SUMMARY

According to a first aspect, there is provided a method of degrading a heterocyclic aldehyde compound comprising the step of treating the heterocyclic aldehyde compound with Enterobacter sp. microorganisms.

Advantageously, the Enterobacter sp. microorganisms may be able to degrade the heterocyclic aldehyde compounds that are present in lignocellulose hydrolysate so as to significantly reduce the amount of the heterocyclic aldehyde compound or to completely remove the heterocyclic aldehyde compounds from the lignocellulose hydrolysate.

Advantageously, the Enterobacter sp. microorganisms may be able to degrade the heterocyclic aldehyde compounds at a higher degradation rate and shorter degradation time as compared to other microorganisms.

Advantageously, the Enterobacter sp. microorganisms may be recycled and reused for a certain number of times without any appreciable loss in their degradation rate.

Advantageously, the degradation may be carried out under aerobic conditions and hence it is not necessary to remove the air from the culture medium when carrying out the degradation of the heterocyclic aldehyde compounds.

Advantageously, the degradation can be carried out in the absence of any nitrogen sources.

According to a second aspect, there is provided a method of degrading a carboxylic acid compound comprising the step of treating the carboxylic acid compound with Bacillus sp. microorganisms.

Advantageously, the Bacillus sp. microorganisms may be able to degrade the carboxylic acid compound that is present in lignocellulose hydrolysate so as to significantly reduce the amount of the carboxylic acid compound or to completely remove the carboxylic acid compounds from the lignocellulose hydrolysate.

Advantageously, the Bacillus sp. microorganisms may be able to degrade the carboxylic acid compound at a higher degradation rate and shorter degradation time as compared to other microorganisms.

Advantageously, the Bacillus sp. microorganisms may be recycled and reused for a certain number of times without any appreciable loss in their degradation rate.

Advantageously, the degradation may be carried out under aerobic conditions and hence it is not necessary to remove the air from the culture medium when carrying out the degradation of the acetic acid compound.

Advantageously, the degradation can be carried out in the absence of any nitrogen sources.

According to a third aspect, there is provided a method of degrading an organic compound in lignocellulose hydrolysate, comprising the step of treating the lignocellulose hydrolysate with at least one of Enterobacter sp. microorganisms and Bacillus sp. microorganisms.

Advantageously, the use of microorganisms to degrade the respective organic compounds from lignocellulose hydrolysate may not generate in any toxic wastes.

### DEFINITIONS

The following words and terms used herein shall have the meaning indicated:

The term “degrading”, when used in connection with a compound, such as a heterocylic aldehyde compound or carboxylic acid compound, refers to the breakage of one or more chemical bonds of said compound.

The terms “treat,” “treatment,” and grammatical variants thereof, when used herein with reference to an organic compound (such as a heterocyclic aldehyde compound or a carboxylic acid compound) refers to contact of the organic compound with a disclosed microorganism which results in degradation or conversion of the organic compound. For example, the treatment may involve degradation of the organic compound so as to convert the organic compound to waste products.

The word “substantially” does not exclude “completely” e.g. a composition which is “substantially free” from Y may be completely free from Y. Where necessary, the word “substantially” may be omitted from the definition of the invention.

Unless specified otherwise, the terms “comprising” and “comprise”, and grammatical variants thereof, are intended to represent “open” or “inclusive” language such that they include recited elements but also permit inclusion of additional, unrecited elements.

As used herein, the term “about”, in the context of concentrations of components of the formulations, typically means +/−5% of the stated value, more typically +/−4% of the stated value, more typically +/−3% of the stated value, more typically, +/−2% of the stated value, even more typically +/−1% of the stated value, and even more typically +/−0.5% of the stated value.

Throughout this disclosure, certain embodiments may be disclosed in a range format. It should be understood that the description in range format is merely for convenience and brevity and should not be construed as an inflexible limitation on the scope of the disclosed ranges. Accordingly, the description of a range should be considered to have specifically disclosed all the possible sub-ranges as well as individual numerical, values within that range. For example, description of a range such as from 1 to 6 should be considered to have specifically disclosed sub-ranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range.

Certain embodiments may also be described broadly and generically herein. Each of the narrower species and subgeneric groupings falling within the generic disclosure also form part of the disclosure. This includes the generic description of the embodiments with a proviso or negative limitation removing any subject matter from the genus, regardless of whether or not the excised material is specifically recited herein.

### DETAILED DISCLOSURE OF EMBODIMENTS

Exemplary, non-limiting embodiments of a method for degrading an organic compound will now be disclosed.

Where the organic compound is a heterocyclic aldehyde compound, the method of degrading the heterocyclic aldehyde compound may comprise the step of treating the heterocyclic aldehyde compound with Enterobacter sp. microorganisms.

The heterocyclic aldehyde compound may have the following formula I:

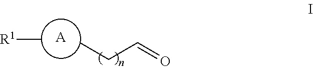

where

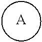

is a 5 to 7 membered heterocyclic ring in which there may be 4 to 5 carbon atoms and 1 to 2 heteroatoms present in the ring;
R¹ is H or hydroxyl-C₁₋₃-alkyl; and
n is an integer from 0 to 2.

In one embodiment,

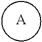

has 4 carbon atoms and 1 heteroatom.

The heteroatom may be N, O or S. In one embodiment, the heteroatom is O.

R¹ may be hydrogen, hydroxymethyl, hydroxyethyl or hydroxypropyl.

n may be an integer selected from 0, 1 or 2. In an embodiment where n is 0. the aldehyde group is directly linked to

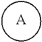

The aldehyde moiety of the heterocyclic aldehyde may have 1 carbon atom (n is 0), 2 carbon atoms (n is 1) or 3 carbon atoms (n is 2).

The heterocyclic aldehyde compound may be at least one of 2-furaldehyde (furfural) and 5-(hydroxymethyl)-2-furaldehyde (HMF), or analogues thereof.

The Enterobacter sp. may be Enterobacter sp. FDS8. The Enterobacter sp. FDS8 may have a 16S rDNA sequence as shown in SEQ ID NO: 3. The inoculation amount of the Enterobacter sp. microorganisms may be in the range selected from the group consisting of about 1 to about 5 g/L, about 2 to about 5 g/L, about 3 to about 5 g/L, about 3 to about 4 g/L and about 4 to about 5 g/L.

The Enterobacter sp. microorganisms may, before being used to treat the heterocyclic aldehyde compound, be cultivated for a time period in the range selected from the group consisting of about 15 to about 50 hours, about 15 to about 20 hours, about 15 to about 30 hours, about 15 to about 40 hours, about 20 to about 50 hours, about 30 to about 50 hours, about 40 to about 50 hours and about 15 to about 17 hours.

The degradation rate of the 2-furaldehyde may be in the range selected from the group consisting of about 100 to about 600 mg/L/h, about 200 to about 600 mg/L/h, about 300 to about 600 mg/L/h, about 400 to about 600 mg/L/h, about 500 to about 600 mg/L/h, about 100 to about 200 mg/L/h, about 100 to about 300 mg/L/h, about 100 to about 400 mg/L/h, about 100 to about 500 mg/L/h, about 500 to about 550 mg/L/h and about 530 to about 540 mg/L/h.

The degradation rate of the 5-(hydroxymethyl)-2-furaldehyde may be in the range selected from the group consisting of about 10 to about 200 mg/L/h, about 50 to about 200 mg/L/h, about 100 to about 200 mg/L/h, about 150 to about 200 mg/L/h, about 10 to about 50 mg/L/h, about 10 to about 100 mg/L/h, about 10 to about 150 mg/L/h and 120 to 130 mg/L/h.

The Enterobacter sp. microorganisms may not require a nitrogen source to be added to the culture medium in order to degrade the heterocyclic aldehyde compound.

After one round of degradation, the method may further comprise the step of collecting the Enterobacter sp. microorganisms. The method may further comprise the step of reusing the collected Enterobacter sp. microorganisms in a further treating step.

The Enterobacter sp. microorganisms may be recycled and reused for at least five times. Hence, the steps of treating the heterocyclic aldehyde compound, collecting the Enterobacter sp. microorganisms and reusing the Enterobacter sp. microorganisms may be repeated for at least one time, for at least two times, for at least three times, for at least four times or for at least five times.

The heterocyclic aldehyde compound may be present in lignocellulose hydrolysate, or any source which requires the heterocyclic aldehyde compound present to be reduced to a negligible amount, such as in wastewater.

Where the organic compound is a carboxylic acid compound, the method of degrading the carboxylic acid compound may comprise the step of treating the carboxylic acid compound with Bacillus sp. microorganisms.

The carboxylic acid may be of the general formula R²COOH, where R² is an alkyl group having 1 to 4 carbon atoms such that the carboxylic acid compound may have 2 to carbon atoms. The carboxylic acid compound may be selected from the group consisting of acetic acid, propanoic acid, butanoic acid, pentanoic acid and analogues thereof.

The Bacillus sp. may be Bacillus sp. ADS3. The Bacillus sp. ADS3 may have a 16S rDNA sequence as shown in SEQ ID NO: 4. The inoculation amount of the Bacillus sp. microorganisms may be in the range selected from the group consisting of about 3 to about 4 g/L, about 3 to about 3.5 g/L, about 3 to about 3.3 g/L and about 3 to about 3.1 g/L.

The degradation rate of the acetic acid by the Bacillus sp. microorganisms may be in the range selected from the group consisting of about 500 to about 600 mg/L/h, about 500 to about 550 mg/L/h, about 550 to about 600 mg/L/h and about 535 to about 545 mg/L/h.

After one round of degradation, the method may further comprise the step of collecting the Bacillus sp. microorganisms. The method may further comprise the step of reusing the collected Bacillus sp. microorganisms in a further treating step.

The Bacillus sp. microorganisms may be recycled and reused for at least three times. Hence, the steps of treating the carboxylic acid compound, collecting the Bacillus sp. microorganisms and reusing the Bacillus sp. microorganisms may be repeated for at least one time, for at least two times or for at least three times.

The carboxylic acid compound may be present in lignocellulose hydrolysate, or any source which requires the acetic acid present to be reduced to a negligible amount.

There is also provided a method of degrading an organic compound in lignocellulose hydrolysate, comprising the step of treating the lignocellulose hydrolysate with at least one of Enterobacter sp. microorganisms and Bacillus sp. microorganisms.

Lignocellulose hydrolysate is an intermediate product during the conversion of lignocellulose to value-added fuels, which requires the hydrolysis of lignocellulose to form lignocellulose hydrolysate.

Lignocellulose can be obtained from any agricultural sources and can include woodchips, saw dust, waste paper pulp, switchgrass (panicum virgatum), miscanthus grass species, corn cobs, corn stover, oil palm empty fruit bunch (EFB), olive husk, coffee bean husk, rice husk, rice straw, spent mushroom compost, palm foliage, palm trunk, palm kernel shells, cotton stalk, palm fiber, farm effluent, slaughterhouse waste, flower cuttings, spent flower compost, wheat straw, rape straw, fruit waste, vegetable waste, wood waste, and the like.

In order to obtain the lignocellulose hydrolysate, the lignocellulose may be subjected to a hydrolysis process as known to a person skilled in the art. For example, the hydrolysis process may be chemical hydrolysis (which requires the use of acid), enzymatic hydrolysis (which requires the use of cellulase enzymes) or steam explosion.

The microorganisms used may be isolated from nature or may be obtained from any commercial sources. Where the organic compound is a heterocyclic aldehyde compound, the Enterobacter sp. microorganisms may be Enterobacter sp. FDS8. Where the organic compound is a carboxylic acid compound, the Bacillus sp. microorganisms may be Bacillus sp. ADS3.

The above microorganisms may be used alone or may be used in a consortium with each other.

There is also provided the use of Enterobacter sp. microorganisms for degrading a heterocyclic aldehyde compound. There is also provided the use of Bacillus sp. microorganisms for degrading a carboxylic acid compound. There is also provided the use of a mixture of Enterobacter sp. microorganisms and Bacillus sp. microorganisms for degrading a mixture of a heterocyclic aldehyde compound and a carboxylic acid compound.

### BRIEF DESCRIPTION OF DRAWINGS

The accompanying drawings illustrate a disclosed embodiment and serves to explain the principles of the disclosed embodiment. It is to be understood, however, that the drawings are designed for purposes of illustration only, and not as a definition of the limits of the invention.

FIG. 1 is a graph showing the degradation of furfural by the isolated colonies.

FIG. 2 is a microscopy image showing the cellular morphology of Enterobacter sp FDS8 at 400× magnification.

FIG. 3a is a graph showing the furfural degradation rate as a result of the pre-cultivation time. FIG. 3b is a graph showing the HMF degradation rate as a result of the pre-cultivation time.

FIG. 4a is a graph showing the degradation rates of furfural () and HMF (□) as a result of the inoculation amount of Enterobacter sp FDS8. FIG. 4b is a graph showing the specific degradation rates of furfural () and HMF (□) as a result of the inoculation amount of Enterobacter sp FDS8.

FIG. 5 is a graph showing the recycle and reuse of the Enterobacter sp FDS8 cells for detoxification of furfural ( [symbol] ) and HMF ().

FIG. 6 is a microscopy image showing the cellular morphology of Bacillus sp. ADS3 at 400× magnification.

FIG. 7 is a graph showing the recycle and reuse of the Bacillus sp. ADS3 cells for detoxification of acetic acid.

### EXAMPLES

Non-limiting examples of the invention and a comparative example will be further described in greater detail by reference to specific Examples, which should not be construed as in any way limiting the scope of the invention.

### Materials

Empty fruit bunch (EFB) of oil palm trees was provided by Wilmar International Limited, Singapore. It was naturally dried and grounded to small particles (<1 mm) followed by oven-drying at 105° C. overnight before use. EFB compositions were analyzed following the standard procedures of NREL¹⁴.

All chemicals used were obtained from Sigma-Aldrich of Missouri of the United States of America.

### Acid-Catalyzed EFB Hydrolysis

A typical procedure for the acid-catalyzed EFB hydrolysis was as follows. 30 g of EFB and 300 ml of tap water containing 0.50 (w/v) of H₂SO₄ and 0.20 (w/v) of H₃PO₄ were added into a 1 L Parr reactor (Fike, Blue Springs, Mo. of the United States of America). The reactor was heated to 160° C. for 30 minutes followed by immediate cooling down to room temperature by circulated cooling water. The solid phase was separated from liquid phase by filtration. The composition of the liquid phase was analyzed by HPLC. The typical composition of the liquid phase was 0.42 g/L HMF, 1.64 g/L furfural and 5.61 g/L acetic acid.

### Analytical Methods

Xylose, glucose, arabinose, acetic acid, furfural and HMF were analyzed by HPLC (LC-10AT, refractive index detector SPD-10A, Shimadzu, Kyoto, Japan) with a Bio-Rad Aminex HPX-87 H column (Bio-Rad, Herculse, Calif., USA) at 30° C. The mobile phase was 5 mM H₂SO₄ at 0.6 ml/min.

### Calculations

The degradation rate of the inhibitors was defined as the amount of inhibitors consumed per liter per hour. The specific degradation rate was defined as the amount of inhibitors consumed per gram (dry weight) of cells per hour.

### Example 1

### Isolation of Enterobacter FDS8

Soil samples (1 g) were dispersed in 100 ml of 0.85% of NaCl solution. The supernatants were collected, diluted and spread onto PDA plates containing furfural. The PDA plates for screening furfural-degrading microbes contained (per liter) 2 g of furfural, 5 g of potato extract, 20 g of glucose and 20 g of agar at a pH of 7.0. The plates were kept in an incubator at 30° C. for 2 to 3 days until the occurrence of clear colonies. The colonies were picked up and cultivated in 40 ml of liquid PD medium for 24 hours. The liquid PD medium for cultivating the furfural-degrading microbes had the same composition with the solid PDA plates except furfural and agar.

5 ml of the cell culture was added into the lignocellulose hydrolysates containing up to 2 g/L of furfural and 0.5 g/L of HMF. The mixture was incubated at 30° C. for two days and liquid samples (1 ml) were regularly taken for HPLC analysis to monitor the degradation of furfural and HMF.

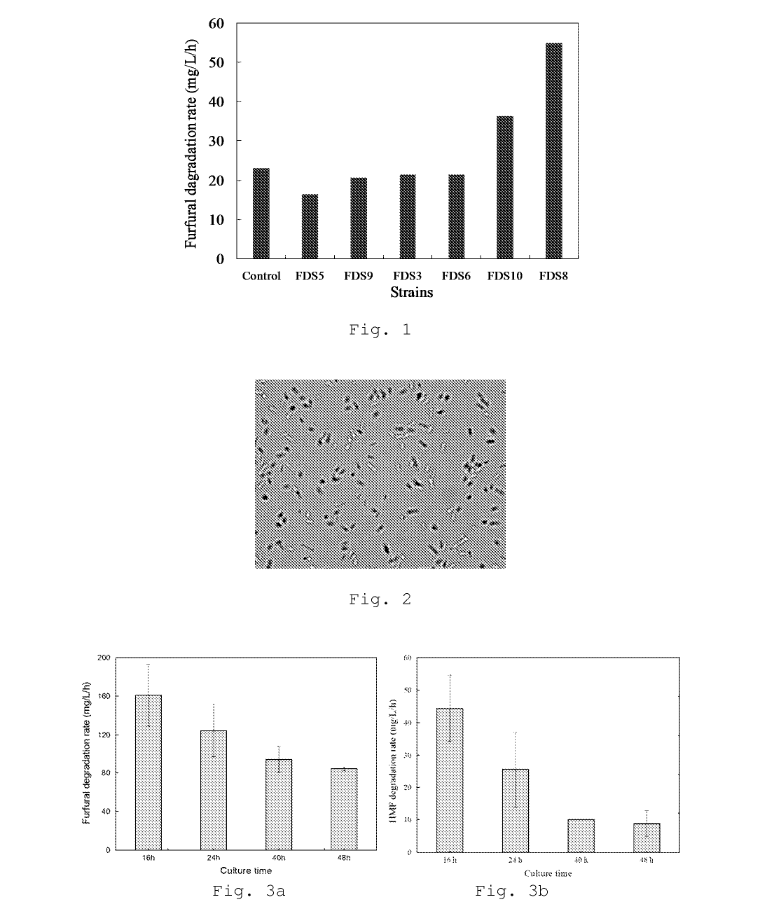

Six colonies were picked up from the PDA plates containing furfural and tested for their ability of degrading furfural (FIG. 1) Among these isolates, FDS8 showed the highest ability of degrading furfural giving a furfural detoxification rate of 2.4 times bigger than that of the control without the isolate. This strain was identified based on the 16S rDNA sequence and used for subsequent experiments. The DNA was extracted using the Promega Wizard® Genomic DNA Purification Kit using manufacturer's protocol.

The 16S rDNA of the selected isolate was amplified by polymerase chain reaction using two primers, 1492R: 5′-GGTTACCTTGTTACGACTT-3′ (SEQ ID NO: 1) and F27: 5′ AGAGTTTGATCCTGGCTCAG-3′ (SEQ ID NO: 2). The composition of the PCR reaction mixture was 5* phusion buffer (20.0 μl), 2 mM dNTP mix (10.0 μl), primer F27 (4.0 μl), primer 1492R (4.0 μl), template genomic DNA (5.0 μl), phusion (finnzymes) (0.6 μl) and water (56.4 μl) making up a total volume of 100.0 μl. The PCR was performed on a Bio-Rad iCycler PCR system with the following program: 1 cycle of 98° C. at 2 minutes; 30 cycles of 98° C. at 0.5 minutes, 42° C. at 0.5 minutes and 72° C. at 2 minutes; 1 cycle of 72° C. at 10 minutes and 1 cycle of 4° C. until removal of the PCR mixture from the iCycler. The PCR sample was sequenced and analyzed with the NCBI nucleotide database to identify the strains.

The 16 S rDNA sequence of the isolate FDS8 was as follows:

(SEQ ID NO: 3)
AGTGGTAAGCGCCCTCCCGAAGGTTAAGCTACCTACTTCTTTTGCAACCC
ACTCCCATGGTGTGACGGGCGGTGTGTACAAGGCCCGGGAACGTATTCAC
CGTGGCATTCTGATCCACGATTACTAGCGATTCCGACTTCATGGAGTCGA
GTTGCAGACTCCAATCCGGACTACGACATACTTTATGAGGTCCGCTTGCT
CTCGCGAGGTCGCTTCTCTTTGTATATGCCATTGTAGCACGTGTGTAGCC
CTGGTCGTAAGGGCCATGATGACTTGACGTCATCCCCACCTTCCTCCAGT
TTATCACTGGCAGTCTCCTTTGAGTTCCCGGCCGGACCGCTGGCAACAAA
GGATAAGGGTTGCGCTCGTTGCGGGACTTAACCCAACATTTCACAACACG
AGCTGACGACAGCCATGCAGCACCTGTCTCACGGTTCCCGAAGGCACTAA
GGCATCTCTGCCAAATTCCGTGGATGTCAAGACCAGGTAAGGTTCTTCGC
GTTGCATCGAATTAAACCACATGCTCCACCGCTTGTGCGGGCCCCCGTCA
ATTCATTTGAGTTTTAACCTTGCGGCCGTACTCCCCAGGCGGTCGACTTA
ACGCGTTAGCTCCGGAAGCCACGCCTCAAGGGCACAACCTCCAAGTCGAC
ATCGTTTACGGCGTGGACTACCAGGGTATCTAATCCTGTTTGCTCCCCAC
GCTTTCGCACCTGAGCGTCAGTCTTTGTCCAGGGGGCCGCCTTCGCCACC
GGTATTCCTCCAGATCTCTACGCATTTCACCGCTACACCTGGAATTCTAC
CCCCCTCTACAAGACTCAAGCCTGCCAGTTTCGAATGCAGTTCCCAGGTT
GAGCCCGGGGATTTCACATCCGACTTGACAGACCGCCTGCGTGCGCTTTA
CGCCCAGTAATTCCGATTAACGCTTGCACCCTCCGTATTACCGCGGCTGC
TGGCACGGAGTTAGCCGGTGCTTCTTCTGCGGGTAACGTCAATCAACACG
GTTATTAACCGTATTGCCTTCCTCCCCGCTGAAAGTGCTTTACAACCCGA
AGGCCTTCTTCACACACGCGGCATGGCTGCATCAGGCTTGCGCCCATTGT
GCAATATTCCCCACTGCTGCCTCCCGTAGGAGTCTGGACCGTGTCTCAGT
TCCAGTGTGGCTGGTCATCCTCTCAGACCAGCTAGGGATCGTCGCCTTGG
TGAGCCATTACCTCACCAACTAGCTAATCCCATCTGGGCACATCCGATGG
CAAGAGGCCCGAAGGTCCCCCTCTTTGGTCTTGCGACATTATGCGGTATT
AGCTACCGTTTCCAGTAGTTATCCCCCTCCATCGGGCAGTTTCCCAGACA
TTACTCACCCGTCCGCCACTCGTCACCCGAGAGCAAGCTCTCTGTGCTAC
CGTTCGACTTGCA

After blasting in NCBI, it was found that the 16S rDNA of the new isolate is most homologous to the bacterial strains Enterobacter sp. ATCC 27981, Enterobacter sp. LMG 5337, Enterobacter sp. ATCC 27990 and Enterobacter sp. ATCC 27982 with a homology of 99.7%, 99.7%, 99.6% and 99.2%, respectively. Therefore, the new isolated was identified as belonging to Enterobacter and named as Enterobacter sp. FDS8, which is a rod-shaped bacterium under the microscope (FIG. 2).

### Example 2

### Optimization of Furfural and HMF Detoxification

### Effect of Pre-Culture Time

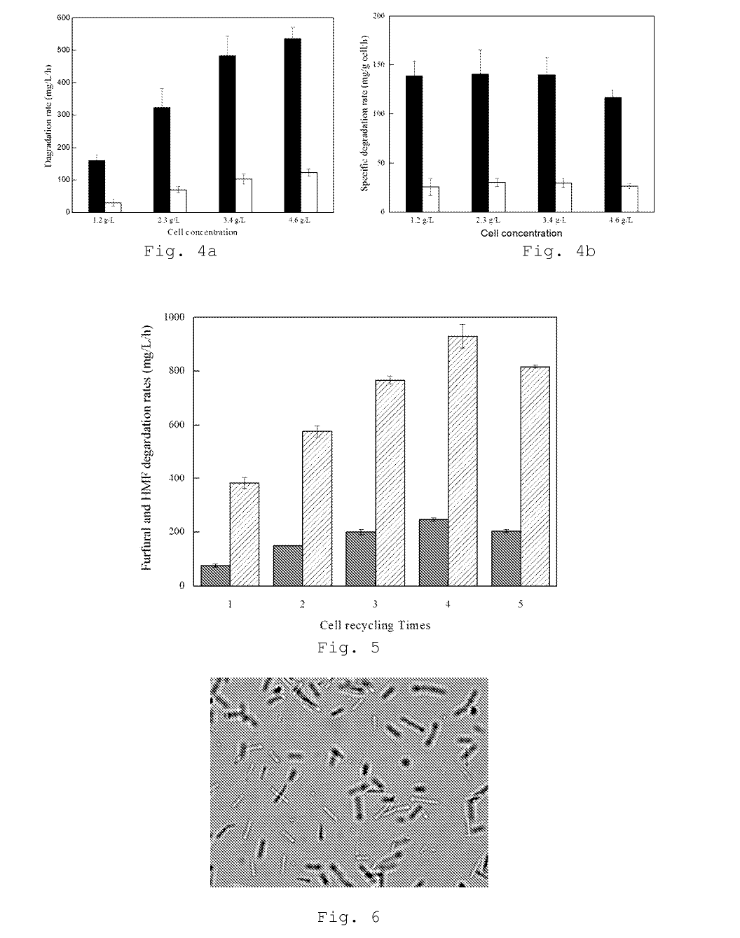

To investigate the effect of pre-cultivation time on detoxification of furfural and HMF, Enterobacter sp. FDS8 was cultivated in the PA liquid medium for 16 to 48 hours before addition of the cells to the lignocelluloses hydrolysate. As shown in FIG. 3a and FIG. 3b, the pre-cultivation time of 16 hours gave the highest furfural (see FIG. 3a) and HMF (see FIG. 3b) degradation rates. Therefore, the pre-cultivation time of 16 hours was used in the subsequent experiments.

In subsequent experiments, Enterobacter sp. FDS8 was cultivated in 40 mL of liquid PA medium for 16 hours. Then mL of the cells were harvested by centrifugation at 10,000 rpm for 10 minutes and added to 20 mL of lignocellulose hydrolysate (having 0.42 g/L HMF, 1.64 g/L furfural and 5.61 g/L acetic acid) in a 250 mL flask. The mixture was incubated at 30° C. with shaking at 150 rpm for a predetermined period of time. The supernatant was collected by centrifugation and analyzed by HPLC. If necessary, after detoxification, the cells were collected by centrifugation and stored at 4° C. for use in the next round of detoxification experiments.

### Effect of Nitrogen Resource

The effect of nitrogen resource on furfural and HMF detoxification rates was investigated (Table 1). The addition of yeast extract, potato extract and two organic nitrogen sources, did not significantly improve the detoxification rates of furfural and HMF as compared to the control without any nitrogen resources, while the addition of a mixture of (NH₄)₂SO₄ and NaNO₃ significantly reduced the detoxification rates of furfural and HMF, in particular, that of furfural. Therefore, in the subsequent experiments, it is not necessary to add any nitrogen source into the lignocellulose hydrolysate, so as to avoid significant consumption of sugars.

TABLE 1
Effect of nitrogen resource

| | Control | | | |
|---|---|---|---|---|
| | (without | Potato | Yeast | |
| | nitrogen | extract | extract | NaNO₃ 5 g/L + |
| | resource) | 10 g/L | 10 g/L | (NH₄)₂SO₄ 5 g/L |
| Furfural | 313 ± 74 | 360 ± 62 | 370 ± 62 | 160 ± 26 |
| degradation | | | | |
| rate (mg/L/h) | | | | |
| HMF | 93 ± 12 | 93 ± 6 | 97 ± 12 | 53 ± 12 |
| degradation | | | | |
| rate (mg/L/h) | | | | |

### Effect of Inoculum Size

Different concentrations of Enterobacter sp. FDS8 cells were added into the lignocelluloses hydrolysates containing furfural and HMF (FIG. 4).

The degradation rates of furfural () and HMF (□) (FIG. 4a) significantly increased with increasing the inoculum amount and became less affected after reaching a certain level. At the inoculum size of 4.6 g/L, the degradation rates of furfural and HMF reached 537 and 123 mg/L/h, respectively.

The specific degradation rates of furfural () and HMF (□) (FIG. 4b) were hardly influenced at lower inoculum size but dropped when the inoculum size increased from 3.4 g/L to 4.6 g/L.

### Recycle and Reuse of the Cells

To verify whether Enterobacter sp. FDS8 cells can be reused in further detoxification assays in order to reduce the process cost, the cells were collected by centrifugation after each round of detoxification experiment and reused in the next round of experiment.

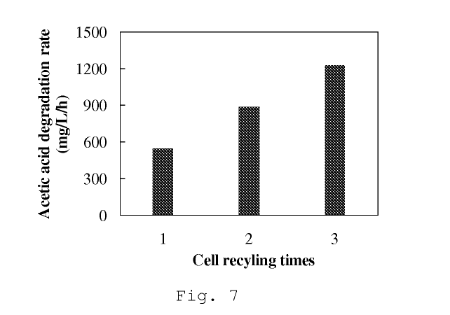

As shown in FIG. 5, the detoxification rates of both furfural ( [symbol] ) and HMF () were increased with the repeated use of the cells and reached the highest at the fourth cycle. Therefore, the cells could be reused for at least 5 cycles in the biological detoxification of furfural and HMF without any decrease in their detoxification ability, as compared to the fresh cells of the first cycle.

### Example 3

### Furfural and HMF Detoxification

(1) 3.4 g/L of Enterobater sp. FDS8 was added into 200 mL of lignocellulose hydrolysate containing 1.64 g/L of furfural and 0.42 g/L of HMF. The mixture was kept at 30° C. for 3 hours. All the furfural and HMF were degraded with a total sugar recovery of 91.1% (Table 2).

TABLE 2
Compositions of lignocellulose hydrolysate before
and after detoxification by Enterobacter sp. FDS8

| | Glucose | Xylose | Arabinose | Acetic | HMF | Furfural |
|---|---|---|---|---|---|---|
| | (g/L) | (g/L) | (g/L) | acid (g/L) | (g/L) | (g/L) |
| 0 h | 1.12 | 18.36 | 1.88 | 5.61 | 0.42 | 1.64 |
| 3 h | 0 | 17.65 | 1.94 | 5.46 | 0 | 0 |

(2) 3.4 g/L of Enterobater sp. FDS8 was added into 20 ml of lignocellulose hydrolysate containing 1.68 g/L of furfural and 0.44 g/L of HMF. The mixture was kept at 30° C. for 3 hours. All the furfural and HMF were degraded with a total sugar recovery of 89.7% (Table 3).

TABLE 3
Compositions of lignocellulose hydrolysate before
and after detoxification by Enterobacter sp. FDS8

| | Glucose | Xylose | Arabinose | Acetic | HMF | Furfural |
|---|---|---|---|---|---|---|
| | (g/L) | (g/L) | (g/L) | acid (g/L) | (g/L) | (g/L) |
| 0 h | 1.73 | 17.57 | 1.68 | 4.72 | 0.44 | 1.68 |
| 3 h | 0 | 16.86 | 1.69 | 6.76 | 0 | 0 |

Based on the experiments above, Enterobacter sp. FDS8 has been shown to be able to efficiently degrade 2 of the 3 major inhibitors, furfural and HMF, usually present in lignocellulose hydrolysate. The furfural and HMF degradation rates respectively reached as high as 537 mg/L/h and 123 mg/L/h (FIG. 4), which are much higher than those (6 to 102 mg/L/h and 1 to 19 mg/L/h, respectively) reported for other microbes (Table 4). Moreover, the Enterobacter sp. FDS8 cells were able to be recycled and reused for at least 5 times without losing their detoxification abilities (FIG. 5). Furthermore, Enterobacter sp. FDS8 was able to degrade furfural and HMF in the absence of nitrogen sources, avoiding the significant consumption of sugars.

TABLE 4
Comparison of detoxification of furfural and HMF
by Enterobacter sp. FDS8 with literature data

| | | | | | Specific | | |
|---|---|---|---|---|---|---|---|
| | | | | | degradation | | |
| | Detoxification | | Degradation | | rate (mg/g | | |
| | temperature | Detoxification | rate (mg/L/h) | | cell/h) | | |
| Microorganism | (° C.) | time (h) | Furfural | HMF | Furfural | HMF | Ref [symbol] |
| Amorphotheca | 25 | 96 | 6 | 9 | N | N | |
| resinae ZN1 | | | | | | | |
| Coniochaeta | 30 | 17 | 74 | 15 | N | N | |
| ligniaria | | | | | | | |
| NRRL30616 | | | | | | | |
| Coniochaeta | 30 | 20 | 54 | 19 | N | N | |
| ligniaria | | | | | | | |
| NRRL30616 | | | | | | | |
| Ureibacillus | 50 | 24 | 15 | 9 | 4 | 3 | |
| thermosphaericus | | | | | | | |
| Issatchenkia | 30 | 24 | 6 | 1 | N | N | |
| occidentalis | | | | | | | |
| CCTCC M | | | | | | | |
| 206097 | | | | | | | |
| Escherichia coli | 37 | 7.5 | 102 | N | N | N | |
| strains KO11 | | | | | | | |
| Enterobacter | 30 | 3 | 537 | 123 | 130 | 40 | [symbol] |
| sp. FDS8 | | | | | | | s [symbol] |

[symbol]

indicates data missing or illegible when filed

### Example 4

### Isolation of Bacillus sp. ADS3

Soil samples (1 g) were dispersed in 100 ml of 0.85% of NaCl solution. The supernatants were collected, diluted and spread onto acetic acid plates containing acetic acid.

The acetic acid plates for screening acetic acid-degrading microbes contained (per liter) 20 g of sodium acetate, 5 g of (NH₄)₂SO₄, 5 g of KNO₃, 2 g of NaH₂PO₄, 0.1 g of MgSO₄.7H₂O, 0.1 g of MnSO₄.7H₂O, 0.1 g of FeSO₄.7H₂O, 1 g of yeast extract and 20 g of agar, at pH 7.0. The plates were kept in an incubator at 30° C. for 2 to 3 days until the occurrence of clear colonies. The colonies were picked up and cultivated in 40 ml of liquid acetic acid medium for hours. The liquid acetic acid medium for cultivating the acetic acid-degrading microbes had the same composition with the solid acetic acid plates except agar.

5 ml of the cell culture was added into the lignocellulose hydrolysates containing 6 g/L of acetic acid. The mixture was incubated at 30° C. for 24 hours and liquid samples (1 ml) were regularly taken for HPLC analysis to monitor the degradation of acetic acid.

One colony, ADS3, was found to be able to effectively degrade acetic acid without obvious consumption of xylose. The DNA of this isolate was extracted using the Promega Wizard® Genomic DNA Purification Kit using manufacturer's protocol and subjected to PCR amplication.

The 16S rDNA of this isolate was amplified by polymerase chain reaction using two primers, 1492R: 5′-GGTTACCTTGTTACGACTT-3′ (SEQ ID NO: 1) and F27: 5′-AGAGTTTGATCCTGGCTCAG-3′ (SEQ ID NO: 2) based on the same PCR recipe as in Example 1. The PCR sample was sequenced and analyzed with the NCBI nucleotide database to identify the strains.

The 16 S rDNA sequence of the isolate ADS3 was as follows:

(SEQ ID NO: 4)
CCTTCGGCGGCTGGCTCCAAAAGGTTACCTCACCGACTTCGGGTGTTACA
AACTCTCGTGGTGTGACGGGCGGTGTGTACAAGGCCCGGGAACGTATTCA
CCGCGGCATGCTGATCCGCGATTACTAGCGATTCCGGCTTCATGTAGGCG
AGTTGCAGCCTACAATCCGAACTGAGAACGACTTTATCGGATTAGCTCCC
TCTCGCGAGTTGGCAACCGTTTGTATCGTCCATTGTAGCACGTGTGTAGC
CCAGGTCATAAGGGGCATGATGATTTGACGTCATCCCCACCTTCCTCCGG
TTTGTCACCGGCAGTCACCTTAGAGTGCCCAACTAAATGATGGCAACTAA
GATCAAGGGTTGCGCTCGTTGCGGGACTTAACCCAACATCTCACGACACG
AGCTGACGACAACCATGCACCACCTGTCACCGTTGTCCCCGAAGGGAAAA
CCATATCTCTACAGTGGTCAACGGGATGTCAAGACCTGGTAAGGTTCTTC
GCGTTGCTTCGAATTAAACCACATGCTCCACCGCTTGTGCGGGCCCCCGT
CAATTCCTTTGAGTTTCAGTCTTGCGACCGTACTCCCCAGGCGGAGTGCT
TAATGCGTTAGCTGCAGCACTAAGGGGCGGAAACCCCCTAACACTTAGCA
CTCATCGTTTACGGCGTGGACTACCAGGGTATCTAATCCTGTTTGCTCCC
CACGCTTTCGCGCCTCAGTGTCAGTTACAGACCAGATAGTCGCCTTCGCC
ACTGGTGTTCCTCCAAATCTCTACGCATTTCACCGCTACACTTGGAATTC
CACTATCCTCTTCTGCACTCAAGTCTCCCAGTTTCCAATGACCCTCCACG
GTTGAGCCGTGGGCTTTCACATCAGACTTAAGAAACCACCTGCGCGCGCT
TTACGCCCAATAATTCCGGACAACGCTTGCCACCTACGTATTACCGCGGC
TGCTGGCACGTAGTTAGCCGTGGCTTTCTAATAAGGTACCGTCAAGGTAC
AGCCAGTTACTACTGTACTTGTTCTTCCCTTACAACAGAGTTTTACGAAC
CGAAATCCTTCTTCACTCACGCGGCGTTGCTCCATCAGGCTTTCGCCCAT
TGTGGAAGATTCCCTACTGCTGCCTCCCGTAGGAGTCTGGGCCGTGTCTC
AGTCCCAGTGTGGCCGATCACCCTCTCAGGTCGGCTACGCATCGTCGCCT
TGGTGAGCCGTTACCTCACCAACTAGCTAATGCGCCGCGGGCCCATCCTA
TAGCGACAGCCGAAACCGTCTTTCAGTATTGTCCCATGAGGGACAATAGA
TTATTCGGTATTAGCCCCGGTTTCCCGGAGTTATCCCAAACTATAAGGTA
GGTTGCCCACGTGTTACTCACCCGTCCGCCGCTAACGTCAAAGGAGCAAG
CTCCTTCTCTGTTCGCTCGACTTGCATGTATAG

After blasting in NCBI, the 16S rDNA of this isolate was found to be 100% identical to that of 14 strains of Bacillus sp. Therefore, this new isolate was identified as belonging to Bacillus and named as Bacillus sp. ADS3, which is a rod-shaped bacterium.

### Example 5

### Recycling and Reuse of Bacillus sp. ADS3

For degradation of acetic acid, Bacillus sp. ADS3 was cultivated in 40 ml of liquid acetic acid medium for 24 hours. The cells were harvested by centrifugation and added to 20 mL of lignocelluloses hydrolysate containing 6 g/L of acetic acid. The mixture was incubated at 30° C. with shaking at 150 rpm for predetermined time periods. The supernatant was collected by centrifugation and analyzed by HPLC. After detoxification, the cells were collected by centrifugation and stored at 4° C. for use in next round of detoxification experiments.

Bacillus sp. ADS3 cells were recycled and reused for 3 times for the detoxification of acetic acid (FIG. 7). Similar to the case of furfural and HMF detoxification using Enterobacter sp. FDS8, the Bacillus sp. ADS3 cells were able to be recycled and reused for at least 3 times with gradually increased detoxification rate.

### Example 6

### Acetic Acid Detoxification

3.1 g/L of Bacillus sp. ADS3 was added into 20 ml of lignocellulose hydrolysate containing 6.9 g/L of acetic acid. The mixture was kept at 30° C. for 17 hours. All of the acetic acid was degraded with a total sugar recovery of 88.4% (Table 5).

TABLE 5
Compositions of lignocellulose hydrolysate
before and after detoxification by Bacillus sp. ADS3

| | Glucose | Xylose | Arabinose | Acetic | HMF | Furfural |
|---|---|---|---|---|---|---|
| | (g/L) | (g/L) | (g/L) | acid (g/L) | (g/L) | (g/L) |
| 0 h | 0 | 17 | 2.34 | 6.87 | 0 | 0 |
| 17 h | 0 | 15.53 | 1.56 | 0 | 0 | 0 |

Based on the experiment above, Bacillus sp. ADS3 has been shown to be able to efficiently degrade one of the 3 major inhibitors, acetic acid, usually present in lignocelluloses hydrolysate. The acetic acid degradation rate of Bacillus sp. ADS3 cells reached as high as 540 mg/L/h (FIG. 7), which is much higher than that reported by other strains (Table 6). Moreover, the Bacillus sp. ADS3 cells were able to be recycled and reused for at least 3 times without losing their detoxification abilities (FIG. 7). Furthermore, Bacillus sp. ADS3 was able to degrade acetic acid in the absence of nitrogen sources, avoiding the significant consumption of sugars.

TABLE 6
Comparison of detoxification of acetic acid by
Bacillus sp. ADS3 with literature data

| | | Detoxi- | | |
|---|---|---|---|---|
| | Detoxification | fication | Degradation | |
| Microorganism | temperature (° C.) | time (h) | rate (mg/L/h) | Reference |
| Amorphotheca | 25 | 96 | 20 | 2 |
| resinae ZN1 | | | | |
| Trichoderma | 30 | 60 | 25 | 1 |
| reesei | | | | |
| Saccharomyces | 30 | 24 | 267 | 11 |
| cerevisiae | | | | |
| Bacillus sp. | 30 | 17 | 540 | This study |
| ADS3 | | | | |

### Example 7

### Consecutive Detoxification of Furfural/HMF and Acetic Acid from Lignocellulose Hydrolysate

Enterobacter sp. FDS8 (3.4 g/L) was added to 20 ml lignocelluloses hydrolysate (containing 1.58 g/L furfural, 0.42 g/L HMF and 4.38 g/L acetic acid), and the mixture was cultured at 30° C. for 3 hours. The Enterobacter sp. FDS8 cells were removed by centrifugation and the Bacillus sp. ADS3 cells (3.1 g/L) were added. The mixture was kept at 30° C. for 17 hours. From Tables 7 and 8, it is clear that the furfural and HMF were completed degraded by Enterobacter sp. FDS8 within 3 hours and acetic acid was completely degraded by Bacillus sp. ADS3 within 17 hours. Therefore, the combined use of the two isolates is expected to be able to significantly reduce the concentrations of these inhibitors in the lignocelluloses hydrolysate. The total sugar recovery was 80.5% after the two-step detoxification (Tables 7 and 8).

TABLE 7
Detoxification of furfural and HMF in
lignocellulose hydrolysate byEnterobacter sp. FDS8 in 3 h

| | Glucose | Xylose | Arabinose | Acetic | HMF | Furfural |
|---|---|---|---|---|---|---|
| | (g/L) | (g/L) | (g/L) | acid (g/L) | (g/L) | (g/L) |
| 0 h | 1.92 | 17.21 | 1.77 | 4.38 | 0.42 | 1.58 |
| 3 h | 0 | 16.9 | 1.85 | 5.21 | 0 | 0 |

TABLE 8
Detoxification of acetic acid in lignocellulose
hydrolysate by Bacillus sp. ADS3 in 17 h

| | Glucose | Xylose | Arabinose | Acetic | HMF | Furfural |
|---|---|---|---|---|---|---|
| | (g/L) | (g/L) | (g/L) | acid (g/L) | (g/L) | (g/L) |
| 0 h | 0 | 17 | 2.34 | 6.87 | 0 | 0 |
| 17 h | 0 | 15.53 | 1.56 | 0 | 0 | 0 |

### Applications

The method of degrading the organic compound (furfural, HMF or acetic acid) may be applicable to all the lignocelluloses-based biorefinery industries such as the bioethanol production industry, or other relevant industries such as the paper making industry.

The method of degrading the organic compound (furfural, HMF or acetic acid) may be a more environmental friendly process since toxic wastes are not produced by the degradative microorganisms.

The organic compound (furfural, HMF or acetic acid) may be degraded to negligible amounts or removed altogether.

The method of degrading the organic compound (furfural, HMF or acetic acid) may be a simple and cost-effective method. The method may not require the use of nitrogen sources and can simply be the addition of the specific microorganisms to the lignocellulose hydrolysate. The method may also not require the use of extremes temperatures or pressures as the method can be undertaken at ambient temperature such as 30° C. and atmospheric pressure.

The method may be a highly efficient way of degrading the organic compound (furfural, HMF or acetic acid) since the degradation rates of the microorganisms disclosed herein that are able to degrade the respective organic compounds are higher than those ever reported.

It will be apparent that various other modifications and adaptations of the invention will be apparent to the person skilled in the art after reading the foregoing disclosure without departing from the spirit and scope of the invention and it is intended that all such modifications and adaptations come within the scope of the appended claims.

### LIST OF REFERENCES

1. Palmqvist E, Hahn-Hägerdal B: Fermentation of lignocellulosic hydrolysates. I: inhibition and detoxification. Bioresource Technology 2000, 74(1):17-24.
2. Zhang J, Zhu Z, Wang X, Wang N, Wang W, Bao J: Biodetoxification of toxins generated from lignocellulose pretreatment using a newly isolated fungus, Amorphotheca resinae ZN1, and the consequent ethanol fermentation. Biotechnol Biofuels 2010, 3:26.
3. Dong H, Bao J: Metabolism: biofuel via biodetoxification. Nat Chem Biol 2010, 6(5):316-318.
4. Okuda N, Soneura M, Ninomiya K, Katakura Y, Shioya S: Biological detoxification of waste house wood hydrolysate using Ureibacillus thermosphaericus for bioethanol production. J Biosci Bioeng 2008, 106(2):128-133.
5. Liu Z L, Slininger P J, Dien B S, Berhow M A, Kurtzman C P, Gorsich S W: Adaptive response of yeasts to furfural and 5-hydroxymethylfurfural and new chemical evidence for HMF conversion to 2,5-bis-hydroxymethylfuran. J Ind Microbiol Biotechnol 2004, 31(8):345-352.
6. Modig T, Liden G, Taherzadeh M J: Inhibition effects of furfural on alcohol dehydrogenase, aldehyde dehydrogenase and pyruvate dehydrogenase. Biochem J 2002, 363(Pt 3):769-776.
7. Zyl Cv, Prior B A, Preez J Cd: Acetic acid inhibition of D-xylose fermentation by Pichia stipitis. Enzyme and Microbial Technology 1991, 13(1):82-86.
8. Nichols N N, Dien B S, Guisado G M, Lopez M J: Bioabatement to remove inhibitors from biomass-derived sugar hydrolysates. Appl Biochem Biotechnol 2005, 121-124:379-390.
9. López M J, Nichols N N, moreno B S D J, Bothast R J: Isolation of microorganisms for biological detoxification of lignocellulosic hydrolysates. Appl Biochem Biotechnol 2004, 64:125-131.
10. Fonseca B G, Moutta Rd O, Ferraz Fd O, Vieira E R, Nogueira A S, Baratella B F, Rodrigues L C, Hou-Rui Z, Silva S Sd: Biological detoxification of different hemicellulosic hydrolysates using Issatchenkia occidentalis CCTCC M 206097 yeast. Journal of Industrial Microbiology & Biotechnology 2011, 38(1):199-207.
11. Schneider. H: Selective removal of acetic acid from hardwood-spent sulfite liquor using a mutant yeast. Enzyme and Microbial Technology 1996, 19(2):94-98.
12. Palmqvist E, Hahn-Hägerdal B, Szengyel Z, Zacchi G, Rèczey K: Simultaneous detoxification and enzyme production of hemicellulose hydrolysates obtained after steam pretreatment. Enzyme and Microbial Technology 1997, 20(4):286-293.
13. Gutierrez T, Buszko M L, Ingram L O, Preston J F: Reduction of furfural to furfuryl alcohol by ethanologenic strains of bacteria and its effect on ethanol production from xylose. Appl Biochem Biotechnol 2002, 98-100:327-340.
14. Yan L, Zhang H, Chen J, Lin Z, Jin Q, Jia H, Huang H: Dilute sulfuric acid cycle spray flow-through pretreatment of corn stover for enhancement of sugar recovery. Bioresource Technology 2009, 100(5):1803-1808.

1. A method of degrading a heterocyclic aldehyde compound comprising the step of treating said heterocyclic aldehyde compound with Enterobacter sp. FDS8 microorganisms.

2. The method of claim 1, wherein the ring structure of said heterocyclic aldehyde compound has 4 to 5 carbon atoms and 1 to 2 heteroatoms.

3. The method of claim 2, wherein the ring structure of said heterocyclic aldehyde compound has 4 carbon atoms and 1 heteroatom.

4. The method of claim 2, wherein said heteroatom is oxygen.

5. The method of claim 1, wherein the aldehyde moiety of said heterocyclic aldehyde compound has 1 to 3 carbon atoms.

6. The method of claim 1, wherein said heterocyclic aldehyde compound is at least one of 2-furaldehyde and 5-(hydroxymethyl)-2-furaldehyde.

7. The method of claim 1, wherein said Enterobacter sp. FDS8 has a 16S rDNA sequence as shown in SEQ ID NO: 3.

8. The method of claim 6, wherein the degradation rate of said 2-furaldehyde is in the range selected from the group consisting of 500 to 600 mg/L/h and 530 to 540 mg/L/h and the degradation rate of said 5-(hydroxymethyl)-2-furaldehyde is in the range selected from the group consisting of 100 to 200 mg/L/h and 120 to 130 mg/L/h.

9. The method of claim 1, wherein the inoculation amount of said Enterobacter sp. FDS8 microorganisms is in the range selected from the group consisting of 3 to 5 g/L, 3 to 4 g/L and 4 to 5 g/L.

10. The method of claim 1, comprising, before said treating step, the step of culturing said Enterobacter sp. FDS8 microorganisms for 15 to 20 hours.

11. The method of claim 1, further comprising the step of collecting said Enterobacter sp. FDS8 microorganisms.

12. The method of claim 11, further comprising the step of reusing the collected Enterobacter sp. FDS8 microorganisms in a further treating step.

13. The method of claim 12, wherein the steps of treating said heterocyclic aldehyde compound, collecting said Enterobacter sp. FDS8 microorganisms and reusing said Enterobacter sp. FDS8 microorganisms are repeated for at least one time.

14. The method of claim 1, wherein said heterocyclic aldehyde compound is present in lignocellulose hydrolysate.

15.-25. (canceled)

26. A method of degrading an organic compound in lignocellulose hydrolysate, comprising the step of treating said lignocellulose hydrolysate with at least one of Enterobacter sp. FDS8 microorganisms and Bacillus sp. microorganisms.

27. The method of claim 26, wherein said organic compound is a heterocyclic aldehyde compound.

28. (canceled)
